(19) United States
(12) Patent Application Publication
(10) Pub. No.: US 2025/0040984 A1
(43) Pub. Date: Feb. 6, 2025

(54) VESSEL HARVESTING DEVICE

(71) Applicants: Terumo Kabushiki Kaisha, Tokyo (JP); Terumo Cardiovascular Systems Corporation, Ann Arbor, MI (US)

(72) Inventor: Yusaku YAMAKI, Kanagawa (JP)

(21) Appl. No.: 18/922,046

(22) Filed: Oct. 21, 2024

Related U.S. Application Data
(63) Continuation of application No. PCT/JP2023/015552, filed on Apr. 19, 2023.

(30) Foreign Application Priority Data
Apr. 21, 2022 (JP) 2022070083

(51) Int. Cl. A61B 18/14; A61B 17/00; A61B 17/29; A61B 18/00

(52) U.S. Cl. CPC A61B 18/1447; A61B 2017/00778; A61B 2017/00969; A61B 2017/2936; A61B 2018/00589; A61B 2018/1455

(57) ABSTRACT

The present invention provides a vessel harvesting device including: a jaw structure mounted on a distal end of a cylindrical body and including an upper jaw and a lower jaw that are opened and closed; and a cutter blade disposed between the upper jaw and the lower jaw and moving in a direction of the axis. The jaw structure includes clamping surfaces of the upper jaw and the lower jaw facing each other, planar electrodes formed on the clamping surfaces, respectively, and an insulating portion formed on at least one of the clamping surfaces and located distal to the planar electrode, the insulating portion being formed of an insulating material. The clamping surface of the upper jaw and the clamping surface of the lower jaw have an inclination angle (θ) that allows contact of only the insulating portion at the distal end when the jaw structure is closed.

### CROSS-REFERENCE TO RELATED APPLICATIONS

Pursuant to 35 U.S.C. § 119(e), this application is a continuation of International Application No. PCT/JP2023/015552, with an international filing date of Apr. 19, 2023, which claims the benefit of Japanese Patent No. 2022-070083 filed on Apr. 21, 2022, the contents of which are hereby incorporated by reference in their entireties.

### TECHNICAL FIELD

The present invention relates to a vessel harvesting device.

### BACKGROUND

In coronary artery bypass grafting (CABG), a blood vessel harvested from a patient is connected so as to bypass a lesion site. The blood vessel to be used is harvested, for example, from the lower limb of a patient. An endoscopic vessel harvesting system (EVH system) is used to harvest a blood vessel.

An endoscopic vessel harvesting system includes an endoscope system, a pneumoperitoneum apparatus, a vessel dissection device, and a vessel harvesting device. The vessel is harvested in such a manner that the vessel dissection device is moved forward along the blood vessel while carbon dioxide gas is supplied by the pneumoperitoneum apparatus, and the blood vessel is dissected from peripheral fatty tissues. Thereafter, the vessel branches branching from the blood vessel are cut while hemostasis is performed by the vessel harvesting device. The vessel branches are cut while being observed with an endoscope. Thereafter, the vessel harvesting device is withdrawn, and the blood vessel is removed from the incision site, whereby the vessel harvesting is completed.

JP 2011-229923 A, for example, discloses an instrument that cuts a tissue under observation with an endoscope.

### SUMMARY

The instrument disclosed in JP 2011-229923 A has electrodes on clamping surfaces of a jaw structure that clamps a tissue. The electrodes heat the tissue by supplying an electric current and stop bleeding. The clamping surfaces are formed with cutter grooves through which a cutter blade passes. The tissue on which hemostasis has been performed is cut by the cutter blade. In the instrument disclosed in JP 2011-229923 A, the clamping surface has a spacer that is an insulator for preventing short circuits between the electrodes.

However, there is a possibility that a fine tissue (for example, fine vessel branch) having a diameter smaller than the clearance formed between the electrodes by the spacer cannot contact the electrodes and cannot be heated. In addition, in a thick blood vessel, a portion where no current flow is generated in the tissue in the vicinity of the spacer, and heating sufficient for hemostasis may not be performed.

An object of the present invention is to solve the problem described above.

One aspect according to the following disclosure provides a vessel harvesting device including: a cylindrical body extending along an axis; a jaw structure mounted on a distal end of the cylindrical body and including an upper jaw and a lower jaw that are opened and closed; and a cutter blade disposed between the upper jaw and the lower jaw and moving in a direction of the axis along cutter grooves of the upper jaw and the lower jaw, in which the jaw structure includes a pair of clamping surfaces formed in a region where the upper jaw and the lower jaw face each other in a closed state, planar electrodes formed on the pair of clamping surfaces, respectively, and an insulating portion formed on at least one of the clamping surfaces and located distal to the planar electrode, the insulating portion being formed of an insulating material, and the pair of clamping surfaces has an inclination angle that allows contact of only the insulating portion when the jaw structure is closed.

The vessel harvesting device according to the above aspect can allow even a thin blood vessel to be in contact with the electrodes, and thus, can reliably stop bleeding even from a thin blood vessel. In addition, the vessel harvesting device can uniformly pass a current even through a thick blood vessel, and thus, can reliably stop bleeding from a thick blood vessel.

### BRIEF DESCRIPTION OF DRAWINGS

FIG. 1 is a configuration diagram of a vessel harvesting system according to an embodiment.

FIG. 2A is a side view of a distal end of a vessel harvesting device in FIG. 1 and a neighboring region thereof, and FIG. 2B is a perspective view illustrating a jaw structure in FIG. 2A from a distal side.

FIG. 3A is a plan view of an upper jaw in FIG. 2A as viewed from a clamping surface, and FIG. 3B is a plan view of a lower jaw in FIG. 2A as viewed from a clamping surface.

FIG. 4A is a side view of an upper jaw assembly in FIG. 2A, and FIG. 4B is a side view of a lower jaw assembly in FIG. 2A.

FIG. 5 is a side view illustrating the jaw structure illustrated in FIG. 2A in a closed state.

FIG. 6 is a perspective view illustrating a cutter blade with the jaw structure being removed from a cylindrical body in FIG. 2A.

FIG. 7A is an explanatory diagram of a marking process of a vessel harvesting method, and FIG. 7B is an explanatory diagram of a process for dissecting a blood vessel with a vessel dissection device.

FIG. 8 is an explanatory diagram of a process for harvesting a blood vessel with the vessel harvesting device illustrated in FIG. 1.

### DESCRIPTION OF EXAMPLE EMBODIMENTS

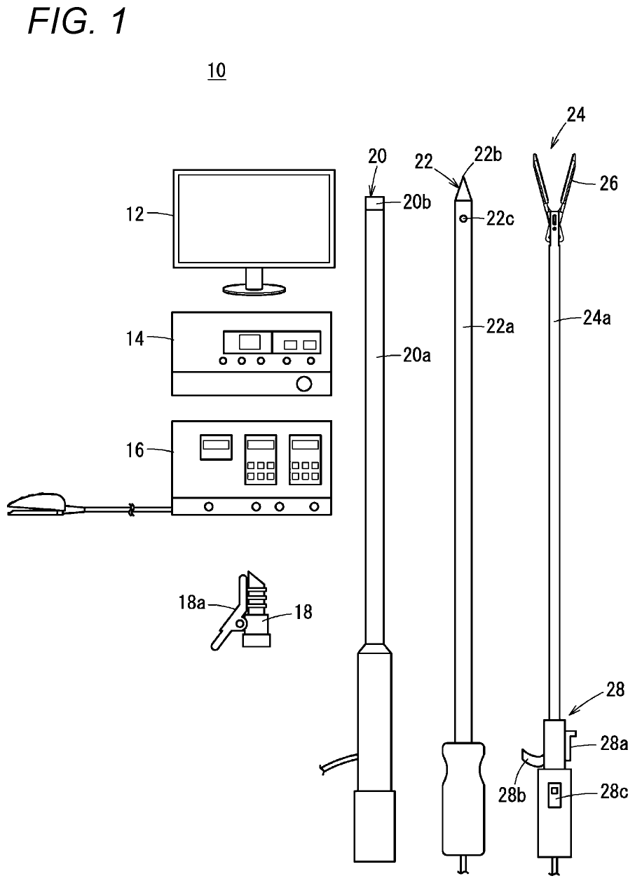

A vessel harvesting system 10 illustrated in FIG. 1 is an endoscopic vessel harvesting (EVH) system used for EVH. The vessel harvesting system 10 includes a display device 12, a high-frequency power source 14, a pneumoperitoneum apparatus 16, a trocar 18, an imaging device 20 (endoscope), a vessel dissection device 22, and a vessel harvesting device 24. Among them, the display device 12 is connected to the imaging device 20. The display device 12 displays an image captured by the imaging device 20. The high-frequency power source 14 supplies high-frequency power to the vessel harvesting device 24 to burn a tissue (a blood vessel 90 or a vessel branch 96). The pneumoperitoneum apparatus 16 supplies carbon dioxide gas to the vessel dissection device 22. The imaging device 20 includes a cylindrical body 20a and a camera 20b attached to a distal end of the cylindrical body 20a. The imaging device 20 is inserted into the body of the patient together with the vessel dissection device 22 or the vessel harvesting device 24 and captures an image of a treated site.

The trocar 18 is inserted into an incision site near a blood vessel. The trocar 18 facilitates introduction of the imaging device 20, the vessel dissection device 22, and the vessel harvesting device 24 into the body. The trocar 18 is fixed to the skin by a clip 18a.

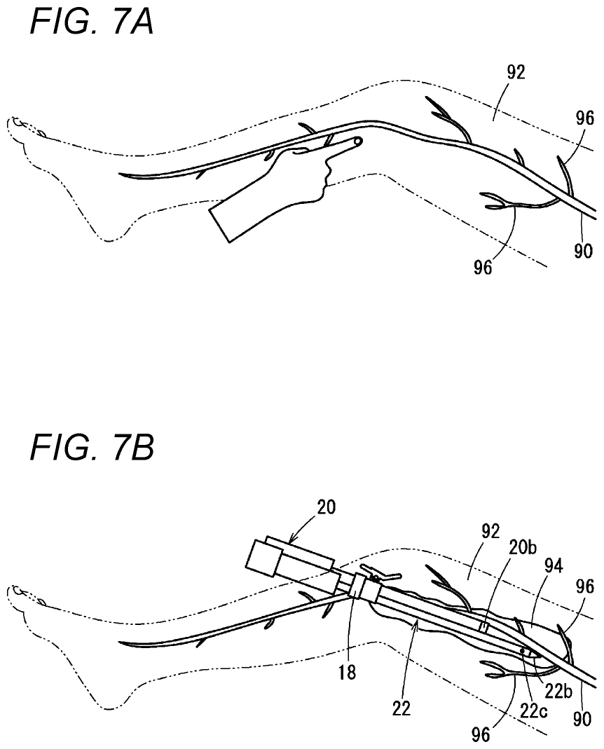

The vessel dissection device 22 includes a cylindrical body 22a and a conical dissection portion 22b attached to a distal end of the cylindrical body 22a. The cylindrical body 22a has an ejection hole 22c for releasing carbon dioxide gas in the vicinity of the distal end. The vessel dissection device 22 dissects the blood vessel 90 and a surrounding tissue 92 around the blood vessel 90 with the dissection portion 22b. The vessel dissection device 22 forms a cavity 94 around the blood vessel 90 by the carbon dioxide gas ejected from the ejection hole 22c (see FIG. 7B).

The vessel harvesting device 24 according to the present embodiment includes a cylindrical body 24a and a jaw structure 26 attached to a distal end of the cylindrical body 24a. The cylindrical body 24a is a cylindrical member extending in the direction of the axis, and accommodates a line (not illustrated) through which high-frequency power flows and an operation wire (not illustrated) or an operation rod (not illustrated) for operating the jaw structure 26.

The vessel harvesting device 24 cuts the vessel branch 96 of the blood vessel 90 that has been dissected by the jaw structure 26. The jaw structure 26 has a function of cutting the vessel branch 96 while burning the vessel branch and stopping bleeding with high-frequency power. The details of the jaw structure 26 will be described later.

The vessel harvesting device 24 has an operation hub 28 at a proximal portion. The operation hub 28 includes a cutter operation portion 28a, a jaw operation portion 28b, and an energization switch 28c. The cutter operation portion 28a performs an operation of moving a later-described cutter blade 34 in the direction of the axis. The jaw operation portion 28b performs an operation of opening and closing the jaw structure 26. The energization switch 28c switches between supply and stop of high-frequency power to the jaw structure 26. The jaw structure 26 specifically has the following configuration.

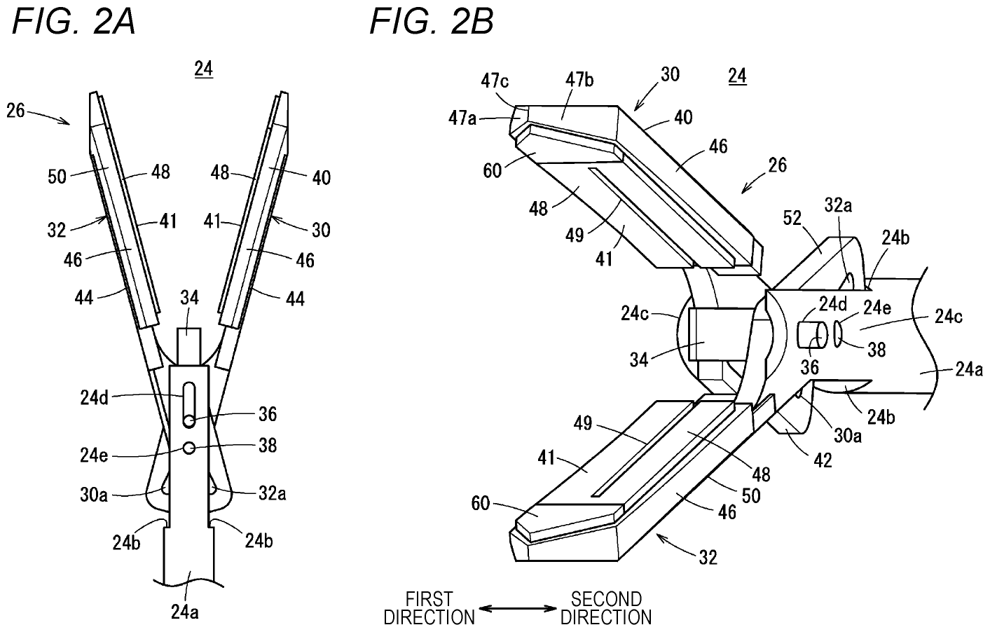

As illustrated in FIG. 2A, the jaw structure 26 is attached to the distal end of the cylindrical body 24a. As illustrated in FIG. 2B, the cylindrical body 24a has, on its distal portion, a pair of recessed grooves 24b formed by cutting out a part of the cylindrical body in the circumferential direction. The pair of recessed grooves 24b is formed at positions separated by 180° in the circumferential direction. Each of the recessed grooves 24b extends in the direction of the axis. The jaw structure 26 is accommodated in the recessed grooves 24b.

The cylindrical body 24a has a pair of support portions 24c extending toward the distal end between the pair of guide grooves 24d. The support portions 24c support the jaw structure 26. Each of the support portions 24c has a guide groove 24d and an opening/closing pin attachment hole 24e. The guide groove 24d is located distal to the opening/closing pin attachment hole 24e. The guide groove 24d extends in the direction of the axis. The opening/closing pin attachment hole 24e has a circular shape. The center positions of the guide groove 24d and the opening/closing pin attachment hole 24e are shifted by 90° in the circumferential direction of the cylindrical body 24a with respect to the center of the recessed groove 24b.

As illustrated in FIG. 2A, the jaw structure 26 includes an upper jaw assembly 30, a lower jaw assembly 32, and the cutter blade 34. The upper jaw assembly 30 and the lower jaw assembly 32 are connected using a shaft pin 36 and an opening/closing pin 38. The shaft pin 36 is fixed with respect to the upper jaw assembly 30 and the lower jaw assembly 32. The shaft pin 36 serves as a rotation center of the upper jaw assembly 30 and the lower jaw assembly 32. The shaft pin 36 is inserted into the guide grooves 24d of the cylindrical body 24a. The guide grooves 24d extend in the direction of the axis and allow the shaft pin 36 to move in the direction of the axis. The shaft pin 36 moves in the guide grooves 24d with displacement of the jaw structure 26 in the direction of the axis.

The opening/closing pin 38 is fixed to the cylindrical body 24a. The opening/closing pin 38 is displaced relative to the upper jaw assembly 30 and the lower jaw assembly 32 as the jaw structure 26 is displaced in the direction of the axis. As illustrated in FIG. 2B, the opening/closing pin 38 is inserted into a first sliding groove 30a of the upper jaw assembly 30 and a second sliding groove 32a of the lower jaw assembly 32. When the jaw structure 26 moves forward or backward in the direction of the axis of the cylindrical body 24a, the opening/closing pin 38 slides in the first sliding groove 30a and the second sliding groove 32a. The upper jaw assembly 30 and the lower jaw assembly 32 rotate according to the position of the opening/closing pin 38 in the first sliding groove 30a and the second sliding groove 32a, and thus, the jaw structure 26 is opened and closed.

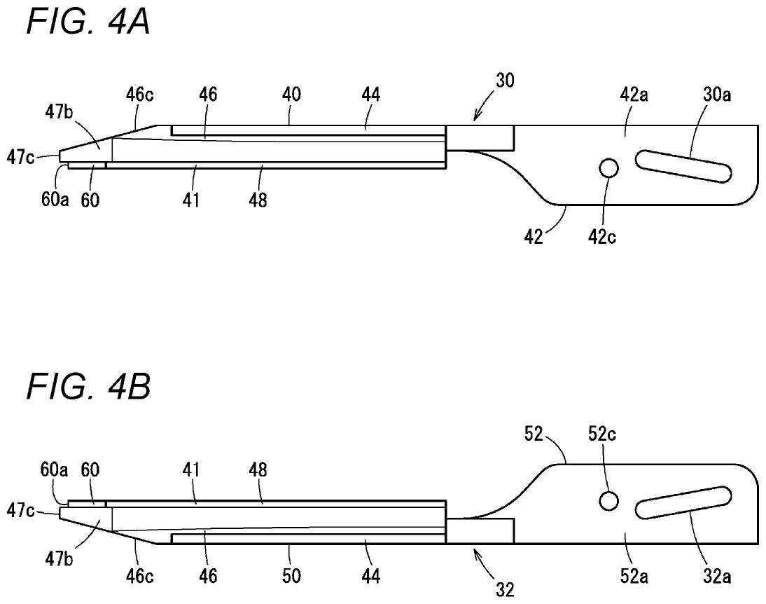

As illustrated in FIG. 4A, the upper jaw assembly 30 includes an upper jaw 40 and a base 42. The upper jaw 40 is located on the distal side and has a clamping surface 41 orthogonal to the rotation direction. The base 42 is located proximal to the upper jaw 40 and is integrally connected to the upper jaw 40. The base 42 has a sliding surface 42a that is flat in a direction orthogonal to the clamping surface 41. The base 42 has a shaft hole 42c and the first sliding groove 30a. The shaft pin 36 is inserted into the shaft hole 42c. The shaft hole 42c is a rotation center of the upper jaw assembly 30. The first sliding groove 30a extends obliquely with respect to the direction of the axis. The opening/closing pin 38 penetrates the first sliding groove 30a.

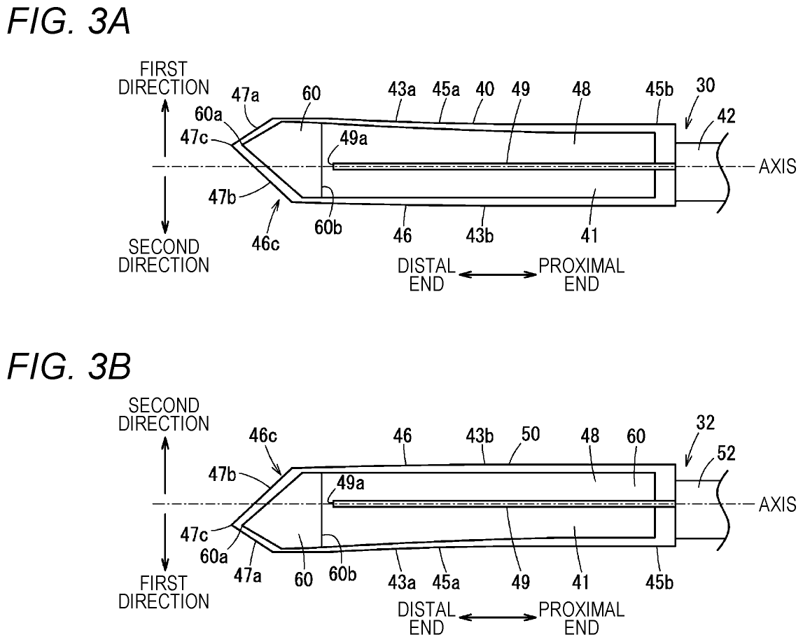

As illustrated in FIGS. 2A and 2B, the upper jaw 40 includes a support body 44, a main body 46, and a planar electrode 48. As illustrated in FIG. 2B, the support body 44 is integrally connected to the base 42, and is formed of the same material (for example, metal) as the base 42. The support body 44 supports the main body 46. The main body 46 is formed of an insulating material such as resin. The main body 46 constitutes most of the upper jaw 40. As illustrated in FIG. 3A, the main body 46 extends while being slightly inclined with respect to the direction of the axis.

As illustrated in FIG. 3A, the upper jaw 40 has a first side surface 43a in a first direction orthogonal to the axis and a second side surface 43b in a second direction opposite to the first direction. The center line of the main body 46 is inclined in the first direction with respect to the axis of the cylindrical body 24a. The first side surface 43a has a curved surface 45a having a gentle arc shape so as to project toward a cutter groove 49. The curved surface 45a of the first side surface 43a has a vertex 45b closest to the cutter groove 49 at the proximal portion. The second side surface 43b extends in parallel with the cutter groove 49 (direction of the axis of the cylindrical body 24a).

The main body 46 has a distal portion 46c protruding from the support body 44 at a distal end thereof. The distal portion 46c has a first inclined surface 47a and a second inclined surface 47b which are inclined with respect to the direction of the axis, and a ridgeline 47c. The first inclined surface 47a is a surface inclined in the first direction, and is adjacent to the first side surface 43a. The second inclined surface 47b is a surface inclined in the second direction, and is adjacent to the second side surface 43b. The ridgeline 47c is formed as a side where the first inclined surface 47a and the second inclined surface 47b meet. The ridgeline 47c is located at the distal end of the upper jaw assembly 30 and extends in a direction orthogonal to the clamping surface 41.

As illustrated in FIG. 3A, the first inclined surface 47a and the second inclined surface 47b meet at an acute angle at the ridgeline 47c. Due to the ridgeline 47c described above, the blood vessel 90 can be preferably dissected from the surrounding tissue 92. In the upper jaw 40, the position of the ridgeline 47c at the distal end is offset from the direction of the axis in the first direction. In the upper jaw 40 in the present embodiment, the position of the ridgeline 47c is close to the position of the first side surface 43a, by which the visibility of the position where the dissection is performed is improved.

As illustrated in FIGS. 2A and 2B, the upper jaw 40 has the clamping surface 41 facing the lower jaw 50. As illustrated in FIG. 3A, a planar electrode 48 and an insulating portion 60 are disposed on the clamping surface 41. The planar electrode 48 is formed of a plate-like metal plate attached to the main body 46. The surface of the planar electrode 48 constitutes a part of the clamping surface 41. The planar electrode 48 is disposed proximal to the insulating portion 60. The insulating portion 60 is formed of a plate-shaped insulating body attached to the main body 46. The insulating portion 60 is located distal to the planar electrode 48 so as to be adjacent thereto. The surface of the insulating portion 60 defines the same plane as the surface of the planar electrode 48. The surface of the insulating portion 60 and the surface of the planar electrode 48 constitute the clamping surface 41. The insulating portion 60 is formed of an insulating material such as resin or ceramic. Note that the insulating portion 60 may be formed integrally with the main body 46. Note that the surface of the insulating portion 60 may protrude further from the surface of the planar electrode 48 as long as the jaw structure 26 can clamp and burn the vessel branch 96 in a closed state. In addition, a clearance may be formed between the insulating portion 60 and the planar electrode 48 as long as the jaw structure 26 can clamp and burn the vessel branch 96 in a closed state.

The clamping surface 41 has the cutter groove 49 extending along the axis. The cutter groove 49 is formed in a region proximal to the distal end of the planar electrode 48. The cutter groove 49 has a length that does not reach the insulating portion 60. A distal-most portion 49a of the cutter groove 49 is positioned proximal to a proximal-most portion 60b of the insulating portion 60 in the direction of the axis. The cutter groove 49 penetrates the planar electrode 48 and reaches the inside of the main body 46. The width of the cutter groove 49 is equal to or slightly larger than the thickness of the cutter blade 34. The cutter groove 49 extends along the axis of the cylindrical body 24a when the jaw structure 26 is closed. The cutter groove 49 guides the movement of the cutter blade 34 in the direction of the axis.

As illustrated in FIGS. 2B and 4B, the lower jaw assembly 32 includes a lower jaw 50 and a base 52. The lower jaw 50 is located at a distal end of the base 52 and has the clamping surface 41 facing the upper jaw 40. The base 52 is located proximal to the lower jaw 50 and is integrally connected to the lower jaw 50. The base 52 has a sliding surface 52a that is flat in a direction orthogonal to the clamping surface 41. The sliding surface 52a slides on the sliding surface 42a of the upper jaw assembly 30. The base 52 has a shaft hole 52c and the second sliding groove 32a. The shaft pin 36 is inserted into the shaft hole 52c. The shaft hole 52c is a rotation center of the lower jaw assembly 32. The second sliding groove 32a extends obliquely in a direction opposite to the first sliding groove 30a. The opening/closing pin 38 penetrates the second sliding groove 32a.

As illustrated in FIGS. 2B and 3B, the lower jaw 50 includes a support body 44, a main body 46, a planar electrode 48, a cutter groove 49, and the insulating portion 60. The lower jaw 50 has a vertically symmetrical shape with respect to the upper jaw 40, and thus, the detailed description of the shape is omitted. The components of the lower jaw 50 same as those of the upper jaw 40 are denoted by the same reference numerals. The lower jaw 50 has the insulating portion 60 on the distal side with respect to the planar electrode 48. The insulating portion 60 of the lower jaw 50 is located in a region facing the insulating portion 60 of the upper jaw 40.

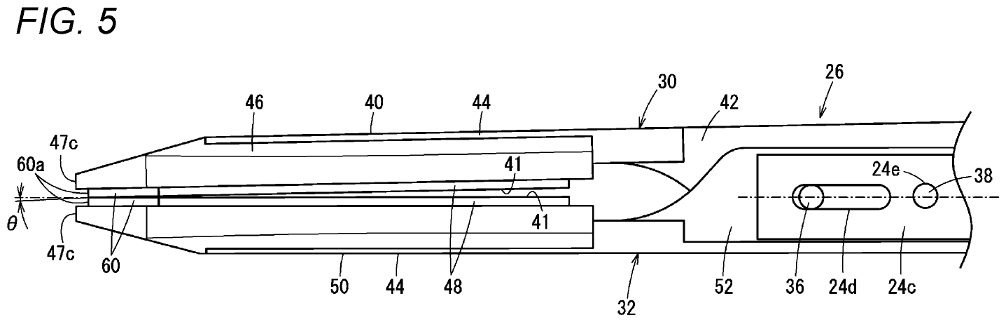

As illustrated in FIG. 2A, the bases 42 and 52 of the upper jaw assembly 30 and the lower jaw assembly 32 are rotatably connected by the shaft pin 36 and the opening/closing pin 38. As illustrated in FIG. 2B, the cutter blade 34 is disposed between the base 42 of the upper jaw assembly 30 and the base 52 of the lower jaw assembly 32. The jaw structure 26 is displaceable in the direction of the axis with respect to the cylindrical body 24a. When the jaw structure 26 is located on the proximal side, the jaw structure 26 is opened, and the upper jaw 40 and the lower jaw 50 are separated as illustrated in FIG. 2A. When the jaw structure 26 is displaced to the distal side in the direction of the axis, the jaw structure 26 is closed as illustrated in FIG. 5. The jaw structure 26 is moved using the jaw operation portion 28b of the operation hub 28 in FIG. 1.

As illustrated in FIG. 5, in a state where the jaw structure 26 is closed, the clamping surface 41 of the upper jaw 40 and the clamping surface 41 of the lower jaw 50 have an inclination angle θ at which a clearance widens toward the proximal side. The upper jaw 40 and the lower jaw 50 come in contact with each other only at the insulating portions 60. The planar electrode 48 of the upper jaw 40 and the planar electrode 48 of the lower jaw 50 are separated with a clearance generated by the inclination angle θ and do not contact each other. Short circuits between the planar electrodes 48 are prevented by the insulating portions 60 formed on the distal side with respect to the planar electrodes 48. More specifically, the jaw structure 26 is configured such that the distal-most portion 60a of the insulating portion 60 of the upper jaw 40 and the distal-most portion 60a of the insulating portion 60 of the lower jaw 50 come in contact with each other. With this configuration, when the jaw structure 26 is closed, the distal-most portions 60a are closed first, and thus, short circuits between the planar electrodes 48 can be prevented.

In the jaw structure 26 according to the present embodiment, the clearance formed between the planar electrode 48 of the upper jaw 40 and the planar electrode 48 of the lower jaw 50 can be made narrower than that in a configuration of having a spacer. Therefore, high-frequency power can be reliably supplied to the thin vessel branch 96. In addition, since the spacer is not provided on the surface of the planar electrode 48, it is possible to uniformly supply high-frequency power to the blood vessel clamped between the planar electrodes 48, whereby it is possible to reliably stop bleeding even from a thick blood vessel.

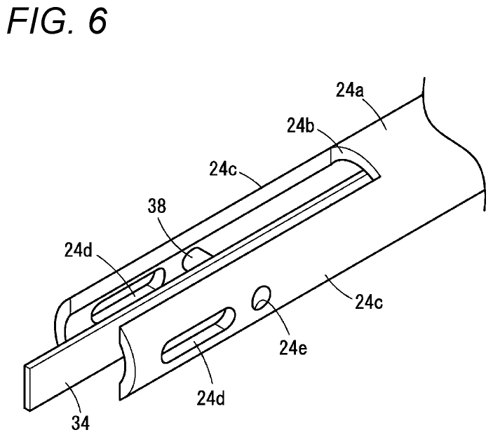

As illustrated in FIG. 6, the cutter blade 34 extends in the direction of the axis of the cylindrical body 24a. The cutter blade 34 can protrude toward the distal end along the direction of the axis by the cutter operation portion 28a of the operation hub 28 illustrated in FIG. 1. The cutter blade 34 is biased toward the proximal side, and is positioned on the proximal side in an initial state as illustrated in FIG. 2B. When the cutter blade 34 is protruded with the jaw structure 26 closed, the cutter blade 34 protrudes along the cutter groove 49 toward the distal end in the direction of the axis, and cuts the vessel branch 96 clamped by the jaw structure 26.

The vessel harvesting device 24 according to the present embodiment is configured as described above. The vessel harvesting system 10 is used for, for example, the following vessel harvesting method.

The vessel harvesting method includes a marking process as illustrated in FIG. 7A. This process includes a step for confirming the position of the saphenous vein at the tibia and a step for putting a mark of approximately 2.5 cm at the position below the knee joint.

Next, the vessel harvesting method proceeds to a process for inserting the trocar 18. During this process, the marked position is incised, and then, the trocar 18 is inserted. The trocar 18 is fixed to the skin by a clip 18a.

Next, the vessel harvesting method proceeds to a vessel dissection process as illustrated in FIG. 7B. During this process, the vessel dissection device 22 and the imaging device 20 are inserted through the trocar 18. This process includes an operation of dissecting the surrounding tissue 92 from the blood vessel 90 with the dissection portion 22b while imaging the blood vessel 90 with the imaging device 20. The blood vessel 90 is dissected with the vessel dissection device 22 while carbon dioxide gas is ejected to the vicinity of the dissection portion 22b from the ejection hole 22c. Through this process, a cavity is formed around the blood vessel 90. After the blood vessel 90 in a predetermined region is dissected from the surrounding tissue, the vessel dissection device 22 and the imaging device 20 are removed from the body.

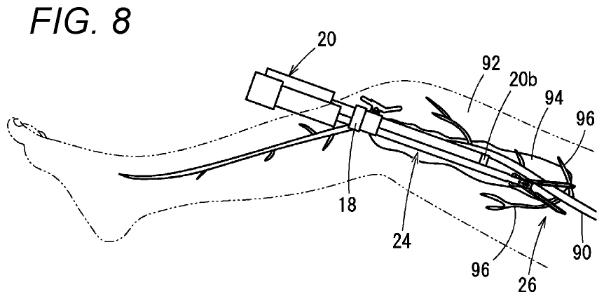

Next, the vessel harvesting method proceeds to a vessel harvesting process as illustrated in FIG. 8. The vessel harvesting process is performed using the vessel harvesting device 24. This process includes a step for cutting the vessel branch 96 with the vessel harvesting device 24. The vessel harvesting device 24 and the imaging device 20 are inserted into the cavity around the blood vessel 90 through the trocar 18. The imaging device 20 is placed on the proximal side with respect to the vessel harvesting device 24 and captures an image of the jaw structure 26 of the vessel harvesting device 24 from the proximal side.

The process for cutting the vessel branch 96 using the vessel harvesting device 24 is performed by the following steps. First, a step for placing the opened jaw structure 26 at the position of the vessel branch 96 under observation with the imaging device 20 is performed. The jaw structure 26 is then closed to clamp the vessel branch 96 between the upper jaw 40 and the lower jaw 50. Then, a step for supplying high-frequency power to the vessel harvesting device 24 is performed. High-frequency power is supplied between the planar electrode 48 of the upper jaw 40 and the planar electrode 48 of the lower jaw 50, and the clamped vessel branch 96 is burned to stop bleeding. Next, a step for cutting the vessel branch 96 is performed by advancing the cutter blade 34 along the cutter groove 49.

Thereafter, an operation of further advancing the vessel harvesting device 24 to cut another vessel branch 96 is performed. In the vessel harvesting device 24 according to the present embodiment, the ridgelines 47c appear at the distal end when the jaw structure 26 is closed. Therefore, during the vessel harvesting process, when a region where the surrounding tissue 92 is not sufficiently dissected is found in a part of the blood vessel 90, the surrounding tissue 92 can be dissected using the ridgelines 47c. The jaw structure 26 is shifted in the first direction with respect to the direction of the axis of the cylindrical body 24a, and the ridgelines 47c are offset from the direction of the axis. Therefore, the vessel harvesting device 24 makes it possible to visually recognize the state near the distal end of the jaw structure 26 with the imaging device 20 located on the proximal side. In addition, the first side surface 43a curved so as to protrude toward the cutter groove 49 further improves the visibility of the vicinity of the distal end by the imaging device 20. In this manner, the vessel harvesting device 24 facilitates the dissection of the remaining surrounding tissue 92.

After cutting of the vessel branch 96 and the blood vessel 90 in the desired region is completed, the vessel harvesting device 24 and the imaging device 20 are withdrawn from the patient's body. Thereafter, the blood vessel 90 is removed from the incision site, whereby the vessel harvesting method is completed.

The vessel harvesting device 24 according to the present embodiment described above is summarized below.

One aspect provides a vessel harvesting device 24 including: a cylindrical body 24a extending along an axis; a jaw structure 26 mounted on a distal end of the cylindrical body and including an upper jaw 40 and a lower jaw 50 that are opened and closed; and a cutter blade 34 disposed between the upper jaw and the lower jaw and moving in a direction of the axis along cutter grooves 49 of the upper jaw and the lower jaw, in which the jaw structure includes a pair of clamping surfaces 41 formed in a region where the upper jaw and the lower jaw face each other in a closed state, planar electrodes 48 formed on the pair of clamping surfaces, respectively, and an insulating portion 60 formed on at least one of the clamping surfaces and located distal to the planar electrode, the insulating portion being formed of an insulating material, and the pair of clamping surfaces has an inclination angle θ that allows contact of only the insulating portion when the jaw structure is closed.

The above-described vessel harvesting device does not have a spacer protruding from the planar electrode, and thus, a clearance between the planar electrodes can be further narrowed, and bleeding from a minute blood vessel can also be stopped by electric heating. In addition, the vessel harvesting device has no spacer that hinders electric heating through the planar electrodes, whereby electric heating can be uniformly performed even on a thick blood vessel. Therefore, the vessel harvesting device can more reliably stop bleeding from the blood vessel.

In the vessel harvesting device, each of the cutter grooves may have a distal end located proximal to the insulating portion and may be formed in a region of the planar electrode. The vessel harvesting device can cut, with the cutter blade, only a blood vessel that contacts the planar electrodes and that is within a region where the blood vessel can be reliably heated with electric heating. The vessel harvesting device can prevent cutting of a blood vessel which is not sufficiently heated by electric heating because it is clamped between the insulating portions and on which sufficient hemostasis is not performed.

In the vessel harvesting device, the inclination angle may generate a clearance between the planar electrode of the upper jaw and the planar electrode of the lower jaw. The vessel harvesting device can prevent short circuits due to contact between the planar electrodes.

In the vessel harvesting device, the surface of the insulating portion may be flush with the surface of the planar electrode. The vessel harvesting device does not include a structure protruding further from the planar electrode, thereby being capable of bringing a thinner blood vessel into contact with the planar electrode of the upper jaw and the planar electrode of the lower jaw. Therefore, the vessel harvesting device can stop bleeding from a minute blood vessel.

Note that the present invention is not limited to the above-described embodiment, and various configurations can be adopted without departing from the gist of the present invention.

1. A blood vessel harvesting device comprising:
a cylindrical body extending along an axis;
a jaw structure coupled to a distal end portion of the cylindrical body and comprising a first jaw and a second jaw that are openable to an open state and closeable to a closed state; and
a cutter blade disposed between the first jaw and the second jaw, the cutter blade being moveable in a direction of the axis along cutter grooves defined by the first jaw and the second jaw,
wherein the first jaw and the second jaw each comprise a clamping surface located where the first jaw and the second jaw face each other while in the closed state,
wherein at least a portion of each clamping surface comprises a planar electrode,
wherein the clamping surface of at least one of the first jaw and the second jaw further comprises an insulating portion located distal to the planar electrode, the insulating portion being formed of an electrically insulating material, and
wherein while the jaw structure is in the closed state: (i) portions of the clamping surfaces of the first jaw and the second jaw are in contact with each other and (ii) the clamping surfaces of the first jaw and the second jaw define an inclination angle therebetween that prevents contact between the planar electrode of the first jaw and the planar electrode of the second jaw.

2. The blood vessel harvesting device of claim 1, wherein the clamping surface of the first jaw and the clamping surface of the second jaw both comprise the insulating portion located distal to the planar electrode.

3. The blood vessel harvesting device of claim 2, wherein the cutter groove defined by the first jaw is defined by the planar electrode of the first jaw and has a distal end located proximal to the insulating portion of the first jaw.

4. The blood vessel harvesting device of claim 3, wherein the cutter groove defined by the second jaw is defined by the planar electrode of the second jaw and has a distal end located proximal to the insulating portion of the second jaw.

5. The blood vessel harvesting device of claim 2, wherein the insulating portions of the first jaw and the second jaw are the portions of the clamping surfaces of the first jaw and the second jaw that are in contact with each other while the jaw structure is in the closed state.

6. The blood vessel harvesting device of claim 2, wherein the insulating portion of the first jaw and the planar electrode of the first jaw are flush with each other to comprise the clamping surface of the first jaw.

7. The blood vessel harvesting device of claim 6, wherein the insulating portion of the second jaw and the planar electrode of the second jaw are flush with each other to comprise the clamping surface of the second jaw.

8. The blood vessel harvesting device of claim 1, wherein the clamping surface of only the first jaw comprises the insulating portion located distal to the planar electrode.

9. The blood vessel harvesting device of claim 8, wherein the cutter groove defined by the first jaw is defined by the planar electrode of the first jaw and has a distal end located proximal to the insulating portion of the first jaw.

10. The blood vessel harvesting device of claim 8, wherein the insulating portion of the first jaw and the planar electrode of the first jaw are flush with each other to comprise the clamping surface of the first jaw.
